(19) United States
(12) Patent Application Publication
(10) Pub. No.: US 2003/0050291 A1
(43) Pub. Date: Mar. 13, 2003

(54) Adrenal enzyme inhibitors

(76) Inventor: Yadon Arad, Mountain View, CA (US)

Correspondence Address: Attn: Malcolm B. Wittenberg, DERGOSITS & NOAH LLP, Suite 1450, Four Embarcadero Center, San Francisco, CA, 94111, US

(21) Appl. No.: 10/280,447

(22) Filed: Oct. 25, 2002

Related U.S. Application Data
(63) Continuation-in-part of application No. 09/880,624, filed on Jun. 12, 2001, now abandoned.

(51) Int. Cl. A61K 31/58

(52) U.S. Cl. 514/172

(57) ABSTRACT

A method of inhibiting adrenal enzyme synthesis in a user in order to improve glucose tolerance, reduce obesity, reduce diabetes, reduce hypertension and reduce atherosclerosis. The preferred active ingredient is the adrenal enzyme inhibitor trilostane or epostane combined with a suitable sustained release carrier which acts as an adrenal enzyme inhibitor and is thus useful for treating diabetes mellitus, hypertension, obesity and atherosclerosis.

### RELATED APPLICATIONS

The present application is a continuation in part of U.S. application Ser. No. 09/880,624 filed on Jul. 15, 2002, which is in turn a divisional of U.S. application Ser. No. 09/880,624 filed on Jun. 12, 2001.

### TECHNICAL FIELD OF THE INVENTION

The present invention involves the use of adrenal enzyme inhibitors for inhibiting increased secretion of cortisol in response to stressful stimuli such as anxiety, anger, depression and fear thus ameliorating such maladies as glucocorticoid-mediated insulin resistance, hypertension, obesity and atherosclerosis.

### BACKGROUND OF THE INVENTION

Diabetes, hypertension, obesity and cardiovascular disease are the most prevalent medical conditions in western societies and are rapidly becoming the major causes of mortality and morbidity in developing countries as well. It is estimated that hypertension affects approximately 20% of the persons in the United States and worldwide. The corresponding numbers for diabetes are 10-15% of the population and for cardiovascular disease 10-15%. Despite major advances in treating these conditions, current treatment is inadequate.

Obesity is likewise becoming more and more prevalent in western societies as well as in developed countries and is now described as an epidemic noting that it is estimated that 10-12% of the population is now considered to be obese. In particular, central or intra-abdominal fat accumulation is associated with increased risk of cardiovascular disease.

These three conditions, diabetes, hypertension and obesity, co-segregate and are associated with a variety of other metabolic abnormalities such as low concentrations of HDL cholesterol, elevated concentrations of triglycerides and small dense low-density lipoprotein.

Decreased blood concentrations of dehydroepiandrosterone and elevated blood concentration of cortisol are associated with increased atherosclerosis. For example, patient's with Cushing's Disease exhibit coronary artery disease at a rate four times higher than the general population. Many conditions associated with increased cortisol secretion, such as depression, low birth weight, advancing age, hostility, and mental stress can predispose one to coronary disease. Other conditions known to be related to the risk of cardiovascular disease such as insulin resistance and central obesity display increased cortisol secretion while stress-related cortisol secretion has been associated with visceral adiposity, high blood pressure and increased concentrations of glucose and insulin. Decrease dehydroepiandrosterone concentrations have been associated with future cardiovascular disease events and angiographically documented coronary disease. Dehydroepiandrosterone feeding prevents atherosclerosis in cholesterol-fed animals.

Subjects with insulin resistance have a similar metabolic profile to patents with Cushing's Disease, who are known to have increased CVD mortality and increase atherosclerosis even years after successful cure. 24-hour cortisol rhysmicity may be responsible, at least in part, for the diurnal variation in glucose tolerance. Mental stress results in elevation of cortisol levels, and this increase is attenuated by estrogen, which is known to prevent atherosclerosis and stress-mediated cortisol secretion was positively correlated with visceral obesity, insulin, glucose and blood pressure. Depressed patients have increase CAD incidence and have increased diurnal plasma concentration of cortisol and this correlates with increased fasting insulin and glucose and increased visceral obesity. Men have a higher rate of cortisol production than women and have a higher incidence of cardiovascular disease. Vital exhaustion and hostility increase adrenal responsiveness to adrenocortical stimulating hormones and hostility is associated with increased coronary calcification, a marker for coronary artery disease.

Glycemic control in diabetic patients deteriorates following even minor everyday stress. Normal morning rise in cortisol inhibits lipolysis and this is reversible by metyrapone. Increased age is associated with increased endogenous glucose production, increased cortisol production, and decreased dehydroepiandrosterone production. Syndromes with insulin resistance, such as polycystic ovary disease, are associated with increased adrenal sensitivity to adrenocortical stimulating hormone during insulin-induced hypoglycemia and increased secretion of cortisol.

Women with increased abdominal fat show increased and prolonged cortisol secretion following mental stress. People with low birth weight, who are known to be at increased risk of insulin resistance, have elevated plasma cortisol concentrations.

The incidence of cardiovascular disease events and the presence of narrowing of the coronary arteries by angiography are associated with decreased concentrations of dehydroepiandrosterone in men and dehydroepiandrosterone supplementation inhibits atherosclerosis in animal models. Metabolic parameters associated with insulin resistance, in particular, central obesity, are associated with evidence of increased coronary artery disease.

11-beta-hydroxysteriod dehydrogenase type 1 knockout mice (which have decreased peripheral conversion of inactive 11-dehydrocortisone to active corticosterone) are resistant to stress-induced hyperglycemia and obesity.

Patients with essential hypertension have increased urinary free cortisol (a marker of increased cortisol production). Blockage of glucocorticoid receptors with RU 486 ameliorated diabetes in OB/OB mice who display massive obesity and diabetes. Glucocorticoids inhibit Glut 4 receptor expression and insulin and non-insulin-induced trans-membrane glucose transport.

Glucocorticoids promote adipose tissue-mediated production of plasminigen activator inhibitor 1 (a protein which promotes the formation of blood clot production by human adipose tissue). Glucocorticoids inhibit the availability of tetrahydropterin and nitric oxide and inhibit flow-mediated vasodilitation, which are normally protective against atherosclerosis. In cell culture, pulse treatment with dexamethasone promotes smooth muscle proliferation, promotes cholesteryl ester formation and impairs cholesterol egress from (HDL) lipoprotein.

Applicant's parent applications taught the use of specific inhibitors of the adrenal enzyme 3-beta-hydroxysteroid dehydrogenase. Examples of such inhibitors include trilostane and epostane. Trilostane, for example, was designed to be used for the treatment of a condition known as Cushing's Syndrome which is caused by excess secretion of cortisol by the adrenal gland. As is noted in applicant's parent applications, unregulated and excessive secretion of cortisol leads to diabetes, hypertension, obesity and atherosclerosis. Notwithstanding the recognized need to control excess cortisol secretion, it is recognized that cortisol is essential to sustain life and its deficiency, known as Addison's Disease can be just as dangerous as excess cortisol secretion.

As further previously noted, stress will increase the secretion of cortisol although it is commonly held among the medical community that an increase in cortisol is physiological and beneficial. However, it is believed by applicant that the recurrent secretion of cortisol is not beneficial and that even in less than pathological concentrations, an increase in cortisol secretion in response to daily stressful stimuli is, in fact, harmful and can be the cause of diabetes, hypertension, obesity and atherosclerosis in those who do not meet the established clinical criteria for Cushing's Disease.

It is thus an object of the present invention to treat such wide ranging maladies as diabetes, hypertension, obesity and atherosclerosis through the time release introduction of an adrenal enzyme inhibitor.

It is yet a further object of the present invention to provide an adrenal enzyme inhibitor in a metered fashion to, on the one hand, not trigger either the clinical criteria for Cushing's Disease on the one hand or Addison's Disease on the other while inhibiting the secretion of cortisol in order to control the release of the subject adrenal enzyme for reducing the occurrence of diabetes, hypertension, obesity or atherosclerosis in a patient.

These and further objects will be more readily appreciated when considering the following disclosure and appended claims.

### SUMMARY OF THE INVENTION

The present invention relates to the use of inhibitors of adrenal synthesis for specific medical conditions. By inhibiting the secretion of glucocorticoids and promoting the secretion of dehydroepiandrosterone by the adrenal gland, this treatment will block or reverse the processes cited above leading to improved glucose tolerance, reduced blood pressure, decreased obesity, in particular, central obesity, and reduced atherosclerosis. The present invention is intended to inhibit the enzyme 3-betahydroxysteriod dehydrogenase through the use of an inhibitor comprising a member selected from the group consisting of epostane and trilostane, a specific inhibitor of 3-beta HSD through a sustained release formulation. Depending upon the severity of the symptoms, doses in the range of 100-1000 mg/day of the inhibitor are to be administered.

### DETAILED DESCRIPTION OF THE INVENTION

As noted in applicant's parent U.S. application Ser. No. 09/880,624 filed on Jun. 12, 2001, it was taught that inhibitors of the adrenal gland synthetic pathway, such as epostane and trilostane, can be delivered transcutaneously, sublingually, orally, rectally or via any other delivery route in order to reduce abnormal secretion of cortisol in response to stress. In doing so, it was recognized that, for the first time, that such inhibitors can be employed to ameliorate glucocorticoids-mediated insulin resistance, hypertension, obesity and atherosclerosis. It was taught that epostane and trilostane could be employed not only for treatment of existing conditions but also for prophylaxis application. It was noted that the inhibitors could be administered in any conventional way and, as such, the parent application taught pharmaceutical compositions including active ingredients in one or more physiological acceptable diluents or carriers. As such, the active ingredients could be formulated for oral, transcutaneous, buccal, sublingual, parenteral, local or rectal administration. Local administration was taught to include administration by insufflation and inhalation. Examples of various types of preparations for local transcutaneous administration include ointments, lotions, crémes, gels, foams, preparations for delivery by transdermal patches, powders, sprays, aerosols, capsules or cartridges for use in an inhalator or insufflator or drops in the form, for example, of eye or nose drops, solutions and suspensions for nebulisation, suppositories, pessaries, retention enemas and chewable or suckable tablets or palettes. It was taught that active ingredients could be contained in a liposome or microcapsulization preparation.

Although the basic chemistry and pharmacology as presented in applicant's parent application remains sound, it has now been determined that improved results can be achieved by administering the suitable adrenal enzyme inhibitor by a mechanism of sustained release. In other words, introducing a spiked dose of an inhibitor for the adrenal enzyme 3-beta hydroxysteroid dehydrogenase may be less effective than was originally hoped and, in fact, may provide for unwanted physiological effects.

As noted previously, Cushing's Syndrome is a disease resulting from the excess secretion of coritsol by the adrenal gland. However, some cortisol is physiologically necessary and, in fact, a deficiency of cortisol, known as Addison's Disease can be just as dangerous as excess cortisol secretion. Applicant's parent application was primarily directed to the use of inhibitors such as trilostane and epostane in an immediate release form in reducing prevailing concentrations of cortisol to achieve the goals stated therein, that is, improving glucose tolerance, reducing blood pressure, reducing obesity and atherosclerosis. However, immediate release of the suitable inhibitor presented several shortcomings which are addressed in practicing the present invention by administering the adrenal enzyme inhibitor through sustained release. Specifically, rapid peaks in the enzyme inhibitor, such as trilostane, resulted in multiple severe side effects, including mouth burning, abdominal cramps, diarrhea and headache. Spiking the dosage also proved to be relatively ineffective since in Cushing's Syndrome, cortisol was secreted not only excessively but also continuously such that by inhibiting secretion intermittently, the patient was left with insufficient concentrations of the adrenal enzyme inhibitor during periods of trough concentrations.

This deficiency is also relevant to the administration of the enzyme inhibitor for the episodic but temporarily unpredictable secretion of cortisol under stressful stimuli. In fact, with immediate release of the enzyme inhibitor it is conceivable that a peak of the inhibitor would occur in the absence of the secretion of cortisol, such as when the patient is not experiencing stress which would result in an Addisonian state while a trough would occur just when the person is exposed to stress thus secreting excess cortisol. Thus, the immediate release formulation of the enzyme inhibitor could be both too toxic as well as ineffective for the treatment of stress-induced cortisol secretion and thus ineffective for the prevention of diabetes, hypertension, obesity and atherosclerosis brought about due to such excessive cortisol secretion. Thus, sustained release of the enzyme inhibitor, such as trilostane and epostane would reduce or eliminate the side effects associated with inappropriately high peaks and eliminate the periods of ineffectiveness due to low troughs.

The time-release delivery systems of use in practicing the present invention are so numerous and well known as to defy characterization. It is contemplated that 100-1000 mg/day of the active inhibitor be administered to a patient together with a suitable time release carrier. For example, encapsulating liposomes can be employed, as well as various resins, such as those used to deliver Ionamin (Phentermine). Drug companies such as Merch & Co., Alza Corporation and Evilsphere Technologies, Inc., actively produce sustain-release carriers which are useful herein. Allen-Bradley offers a fluid bed granulator which applies alternating coatings of active and inert ingredients to tablets that provide time-release medication. Time release carriers can also be based upon their biodegradability, such as disclosed in U.S. Pat. No. 5,900,408, the disclosure of which is incorporated by reference herein. Other patents which claim, or at least disclose, compositions for the time release of pharmaceutical compositions and thus would be useful in practicing the present invention include U.S. Pat. Nos. 6,306,428, 5,830,883 and 5,460,798, the disclosures of which are incorporated by reference herein.

The efficacy of the present invention was confirmed. Specifically, patients were selected in which the secretion of cortisol was suppressed by the ingestion, on the previous evening, of dexamethasone, a potent analog of cortisol that does not cross-react in biochemical assays. Dexamethasone inhibits the endogenous secretion of Adrenocortical Stimulating Hormone (ACTH) by the pituitary gland, thus stopping production of cortisol. The next morning, patients were injected with synthetic ACTH and the response of the adrenal gland, expressed by the secretion of cortisol one hour later, was assayed. In addition, blood pressure was measured, central (intra-abdominal) obesity was measured with a CT scan, and a two-hour glucose tolerance test performed. Pre-clinical atherosclerosis was measured with an electron beam CT scan which quantifies the amount of calcium in the walls of the coronary arteries. It was found that the amount of cortisol secreted in response to synthetic ACTH (a simulation of physiological stress) correlated positively with the blood pressure, degree of obesity, hyperglycemia, resistance to the action of insulin, and with increase atherosclerosis.

I claim:

1. A method of improving glucose tolerance in a user comprising
administering through sustained release, in a suitable sustained release carrier, an adrenal enzyme inhibitor to said user in amounts which provide said improved glucose tolerance.

2. A method of reducing blood pressure in a user comprising administering through sustained release, in a suitable sustained release carrier, an adrenal enzyme inhibitor to said user in amounts which provide said reduced blood pressure.

3. A method of reducing obesity in a user comprising administering through sustained release, in a suitable sustained release carrier, an adrenal enzyme inhibitor to said user in amounts which provide said reduced obesity.

4. A method of reducing atherosclerosis in a user comprising administering through sustained release, in a suitable sustained release carrier, an adrenal enzyme inhibitor to said user in amounts which provide said reduced atherosclerosis.

5. The method of claims 1, 2, 3 or 4 wherein said adrenal enzyme inhibitor is a member selected from the group consisting of trilostane and epostane.

6. The method of claim 1 wherein said adrenal enzyme inhibitor be administered in amounts between 100-1000 mg/day.
